(19) United States
(12) Patent Application Publication
(10) Pub. No.: US 2015/0088006 A1
(43) Pub. Date: Mar. 26, 2015

(54) METHOD FOR DETERMINING AEROBIC CAPACITY

(71) Applicant: Simbionics, Boston, MA (US)

(72) Inventors: Benjamin I. RAPOPORT, New York, NY (US); Craig MERMEL, Brighton, MA (US)

(73) Assignee: SIMBIONICS, Boston, MA (US)

(21) Appl. No.: 14/145,042

(22) Filed: Dec. 31, 2013

Related U.S. Application Data
(60) Provisional application No. 61/880,528, filed on Sep. 20, 2013.

(51) Int. Cl. A61B 5/00; A61B 5/11; A61B 5/0205

(52) U.S. Cl. CPC A61B 5/7225; A61B 5/0205; A61B 5/1118
USPC 600/484

(57) ABSTRACT

A method of estimating the maximal oxygen uptake of an individual on the basis of heart rate data, biometric data, biomechanical data, and geophysical data is described. These data can be collected as the individual engages in activities requiring various levels of exertion, without modifying those activities from the ordinary manner in which they are performed. In particular, in some embodiments the method described here obviates the conventional need for a laboratory setting when estimating maximal oxygen uptake and the method can be applied to estimate maximal oxygen uptake under more natural conditions than conventional testing protocols requiring treadmills or stationary ergometers typically permit. Furthermore, it is described how such estimates of maximal oxygen uptake can be used to estimate other quantities of interest, including fat and carbohydrate metabolism, lactate production, and water and electrolyte loss during exercise.

This application claims priority under 35 U.S.C. §119(e) to U.S. Provisional Application No. 61/880,528, entitled “Method for Determining Aerobic Capacity”, filed Sep. 30, 2013, the contents of which are incorporated by reference herein.

All cited references are incorporated herein in their entirety.

### BACKGROUND

The ability of the body to deliver oxygen to its vital organs and tissues, and the ability of those organs and tissues to consume oxygen in the processes of oxidative cellular metabolism, are fundamental to sustaining life in humans and many other species.

At a macroscopic scale, the delivery of oxygen to organs and tissues of the body relies on the lungs, the heart and blood vessels (together comprising the cardiovascular system) and on the blood itself. The heart pumps blood through the lungs, where blood absorbs oxygen. Oxygen-rich blood then returns to the heart, from which it is pumped through the blood vessels that distribute it to the organs and tissues of the body. Tissues absorb oxygen carried by the blood and use the oxygen in the chemical reactions of oxidative metabolism (also known as “aerobic metabolism”), which provide energy for many essential biological functions.

The rate at which a body consumes oxygen at a given point in time is referred to in the art as the \dot{V}O₂, where the symbol V refers to volume and the dot above the V signifies a rate of change with respect to time, so that the symbol \dot{V}O₂ therefore refers to a volumetric flow of oxygen into the tissues of the body. (Gas volumes are typically assumed to be measured at standard temperature and pressure, so that gas volume can be taken to specify a precise molar quantity.) The quantity \dot{V}O₂ is thus a well defined quantity; in the art this quantity is referred to by a variety of terms under various circumstances. In the present disclosure, it will primarily be referred to as “oxygen uptake.”

As a numeric quantity, \dot{V}O₂ measures the overall rate at which the body is engaged in oxidative metabolism.

Since power refers to a rate of energy expenditure, the rate of oxygen consumption, which is directly related to the rate of oxidative metabolic energy expended in aggregate by the cells of the body, is related directly to the aerobic power output of the body. In the interest of controlling for differences in body size, \dot{V}O₂ is typically reported for a given individual in terms of oxygen volume (at conditions of standard temperature and pressure) per unit time per unit body mass (as in milliliters of oxygen per kilogram body mass per minute). The magnitude of the aerobic power output depends not only on the status of the blood and cardiovascular system, but also on the current demands of the body itself and its systems for energy, which may differ greatly, for example, between states of sleep and vigorous exercise.

In assessing the health or fitness of a given individual, from the perspectives of metabolism (energy production) and cardiovascular status, \dot{V}O₂ must therefore be interpreted with respect to any activity being performed by the body. On the other hand, the maximum \dot{V}O₂ achievable by a given individual is, in principle, dependent only on the metabolic and cardiovascular status of that individual. Maximum \dot{V}O₂, which is known in the art by a variety of names (including “aerobic capacity”), is thus of considerable practical use in the assessment of cardiovascular and metabolic health and fitness. In particular, from the standpoint of health and medicine, exercise capacity as quantified by maximum \dot{V}O₂ has been validated as among the most powerful predictors of mortality associated with cardiovascular disease. Myers, J., et al., Exercise Capacity and Mortality among Men Referred for Exercise Testing, New England Journal of Medicine Vol. 346, pp. 793-801 (2002); Earnest, C. P., et al., Maximal Estimated Cardiorespiratory Fitness, Cardiometabolic Risk Factors, and Metabolic Syndrome in the Aerobics Center Longitudinal Study, Mayo Clinic Proceedings, Vol. 88(3), pp. 259-270 (2013);. Lavie, et al., Impact of Cardiorespiratory Fitness on the Obesity Paradox in Patients With Heart Failure, Mayo Clinic Proceedings, Vol. 88(3), pp. 251-258 (2013). From another perspective, maximum \dot{V}O₂ is of interest to competitive athletes and those who advise them, as it is a strong predictor of performance ability in many domains of sport. Brooks, et. al., Exercise Physiology: Human Bioenergetics and its Applications (2004) 4^{th} Ed. 2005; McArdle W. D., et al., Exercise Physiology, Lippincott Williams & Wilkins (2009) 7^{th} Ed. 2010.

Another parameter, the time constant of heart rate recovery after exercise, k, also has been demonstrated to predict cardiovascular fitness. Wang L., et al., Time constant of heart rate recovery after low level exercise as a useful measure of cardiovascular fitness, Conf. Proc. IEEE Eng. Med. Biol. Soc, Vol. 1, pp. 1799-802 (2006).

In both medical and athletic settings, maximum \dot{V}O₂ is traditionally measured using staged exercise protocols. In schemes such as the widely used Bruce Protocol (Bruce, R. A., et al., Exercising Testing in Adult Normal Subjects and Cardiac Patients, Pediatrics, Vol. 31(4), pp. 742-756 (1963); Bruce, R. A., et al., Maximal Oxygen Intake and Nomographic Assessment of Functional Aerobic Impairment in Cardiovascular Disease, American Heart Journal, Vol. 85(4), pp. 546-562 (1973)), for example, cardiac function may be monitored using electrocardiography, and respiratory volumes as well as oxygen and carbon dioxide gas exchanges may be monitored using clinical spirometry. While such physiologic parameters are measured, an individual patient or athlete is monitored while engaged in standardized forms of exercise (such as treadmill walking or running, or cycle ergometry) at intensities that may be increased in controlled fashion by varying speed, incline, resistance, or other parameters, in a stepwise fashion and at predetermined intervals, until the subject is unable to tolerate further increments in intensity. The point of exhaustion or termination of the test is typically considered the point at which maximum \dot{V}O₂ has been reached, and the corresponding rate of oxygen consumption, determined by clinical spirometry, is then identified as the maximum \dot{V}O₂.

A variety of “sub-maximal” protocols for estimating maximum \dot{V}O₂ have also been described, in which testing stops short of the exhaustion point, and extrapolation methods are used to estimate maximum \dot{V}O₂ on the basis of physiologic data obtained at exercise intensities below that which would elicit exhaustion or maximal oxygen uptake. Observed heart rate and predicted maximum heart rate are common surrogate parameters used in such submaximal protocols. McArdle, W. D., et al., Exercise Physiology, Lippincott Williams & Wilkins (2010).

It will be clear to those skilled in the art how estimates of maximal oxygen uptake can be used in combination with measurements of exercise intensity and duration to estimate other metabolic quantities of interest, including fat and carbohydrate metabolism, lactate production, and water and electrolyte loss during exercise. Brooks, et. al., Exercise Physiology: Human Bioenergetics and its Applications (2004); Rapoport, B. I., Metabolic Factors Limiting Performance in Marathon Runners, Public Library of Science Computational Biology, Vol. 6(10), e1000960 (2010).

The state of the art includes some systems and methods for assessing cardiovascular and aerobic fitness during “free,” unconstrained modes of exercise, as disclosed, for example, by Seppanen and colleagues. Seppanen, et al., Fitness Test, U.S. Pat. Pub. No. 2011-0040193 (2008). However, such systems are unable to account for important physiologic dynamics, and require component methods for eliminating physiologic data captured during periods of non-steady-state physical activity; as such, they do not differ fundamentally from traditional, fixed-protocol physiologic assessments involving assessments through a sequence of physiologic plateaus. The present disclosure describes systems and methods that use mathematical models of physiologic dynamics to enable determination and tracking of aerobic capacity and related physiologic parameters from data continuously acquired during natural activities.

Maximal oxygen uptake is a fundamental indicator of cardiovascular function in both health and disease, of interest to athletes and recreational exercisers as a measure of cardiovascular fitness, and to medical professionals and patients as a predictor of morbidity and mortality from cardiac causes. Existing methods of determining maximal oxygen uptake rely on contrived, fixed, laboratory-based, stepwise exercise protocols; they are time- and resource-intensive, and thus impractical to administer serially to monitor progress; and they typically do not perfectly simulate the natural activities they are designed to reflect.

### SUMMARY

The novelty of the approach presented in this disclosure lies in its ability to estimate oxygen uptake dynamically, without the need for fixed protocols, while individuals engage in natural activities; and to make such measurements repeatedly, as frequently as desired or even continuously, unobtrusively and at minimal marginal cost, outside of a laboratory setting.

The present disclosure provides methods and systems to overcome certain common drawbacks and limitations in the way peak and maximal oxygen uptake are typically measured and monitored over time. This disclosure introduces a “natural experiment” construct that permits maximal oxygen uptake to be estimated on the basis of biometric, mechanical, and geophysical data collected during natural activities, without the need for cumbersome laboratory equipment or artificially controlled conditions. This disclosure also introduces a dynamic model of cardiovascular physiology that accounts for continuously changing oxygen demands of muscle tissue in response to fluctuations in power required by natural activities, thereby making it possible to estimate oxygen uptake under non-steady-state physiologic conditions, without protocols specifically designed to achieve successive steady-state plateaus in oxygen uptake.

In one aspect, the present disclosure relates to the estimation of maximal oxygen uptake (aerobic capacity) in individuals engaging in walking, hiking, running, bicycling, and other activities associated with aerobic exercise.

In another aspect, the present disclosure relates to monitoring the trajectory of the aerobic capacity of an individual through repeated estimations of his or her maximal oxygen uptake, derived from data acquired at successive points in time.

In another aspect, the present disclosure relates to monitoring the trajectories of aerobic capacity of populations of individuals through repeated estimations of the maximal oxygen uptake of large groups of individuals (derived from data acquired at successive points in time), and through statistical analysis determining the demographic variables and activity patterns associated with value ranges and systematic changes in maximal oxygen uptake.

In another aspect, the present disclosure relates to the use of “natural experiments” conducted in large populations to refine and improve the statistical methods used for estimating oxygen uptake and similar biometric functions, and for predicting changes in maximal oxygen uptake and other performance metrics over time.

In one aspect, the present disclosure relates to a computerized method for dynamically measuring maximal oxygen uptake of a user during aerobic activity. In some aspects, the method can include (a) electronically measuring instantaneous heart rate data, instantaneous biomechanical data, and instantaneous geophysical data of the user over a period of time, using one or more sensors; (b) setting an oxygen uptake model for the user and storing the oxygen uptake model in memory of a computer; (c) determining, using the computer, a maximum heart rate of the user and storing the maximum heart rate in memory; (d) determining, using a computer, a plurality of instantaneous oxygen uptake estimates over the period of time based on the oxygen uptake model, the maximum heart rate, instantaneous biomechanical data, instantaneous geophysical data, and instantaneous incline data; and (e) determining, using a computer, a heart rate relaxation constant and a maximal oxygen uptake of the user based at least in part on the plurality of instantaneous oxygen uptake estimates, the maximum heart rate, instantaneous heart rate data, instantaneous biomechanical data, and instantaneous geophysical data.

In some embodiments, the aerobic activity can include running, walking, hiking, cycling, cross-country skiing, swimming or stair climbing. In some embodiments, the user can be a mammal, a human, a horse or a dog. In some embodiments, determining a heart rate relaxation constant (k) and a maximal oxygen uptake can include determining the following relationship:

$$\frac{d R ( t )}{d t} = k ( {R}_{ma x} \frac{{P}_{demand} ( t )}{\dot{V} {O}_{2 m ax}} - R ( t ) ) ,$$

wherein R(t) can be heart rate at a given time, Rₘₐₓ can be the maximum heart rate of the
user and P_{demand}(t) can be a power demanded at a given time.

In some embodiments, the biomechanical data can include instantaneous speed of the user. In some embodiments, the geophysical data can include instantaneous incline, latitude, longitude and altitude data. In some embodiments, the one or more sensors can include a heart rate monitor, a global positioning sensor, and a gyroscope. In some embodiments, the method can include repeating steps (a)-(e) over a plurality of periods of time. In some embodiment, the method can include performing steps (a)-(e) for a plurality of users. In some embodiments, the heart rate relaxation constant can be a numerical parameter that measures a rate at which the heart rate of a user changes in response to oxygen demand by body tissues.

Another aspect of the present disclosure relates to a computerized method for dynamically measuring maximal oxygen uptake of a user during aerobic activity. In some embodiments, the method can include (a) electronically measuring instantaneous physiologic data, instantaneous biomechanical data, and instantaneous geophysical data of the user over a period of time, using one or more sensors; (b) using a computer to create physiologic, biomechanical, and geophysical time-series data based on the instantaneous physiologic data, instantaneous biomechanical data, and instantaneous geophysical data; (c) establishing, using the computer, an oxygen uptake model for the user; (d) estimating, using the computer, a plurality of instantaneous oxygen uptake values over the period of time based on the physiologic, biomechanical, and geophysical data obtained by the sensors; (e) predicting, using the computer, a maximum oxygen uptake of the user based on the oxygen uptake model, the oxygen uptake values and the physiologic, biomechanical, and geophysical time-series data.

### BRIEF DESCRIPTION OF THE DRAWINGS

FIG. 1 is a block diagram overview of a system for estimating maximal oxygen uptake of an individual engaged in physical activity, according to embodiments of the present disclosure.

FIG. 2 is a block diagram overview of a system for estimating maximal oxygen uptake of a group of individuals engaged in physical activity, simultaneously or asynchronously, according to embodiments of the present disclosure.

FIG. 3 is a flow chart illustrating a method for estimating maximal oxygen uptake during physical activity, utilized by the system shown in FIGS. 1-2, according to embodiments of the present disclosure.

FIG. 4 is a flow chart illustrating a sub-method, for acquiring and storing biometric and anthropometric data from a user, utilized within the system for estimating maximal oxygen uptake during physical activity (shown in FIGS. 1-2) according to embodiments of the present disclosure. This flow chart corresponds to block 305 of FIG. 3.

FIG. 5 is a flow chart illustrating a sub-method, for selecting the type of activity being performed by a user, together with the appropriate variables and sensors required to monitor that activity, utilized within the system for estimating maximal oxygen uptake during physical activity (shown in FIGS. 1-2) according to embodiments of the present disclosure. This flow chart corresponds to block 310 of FIG. 3.

FIG. 6 is a flow chart illustrating a sub-method, for acquiring and storing heart rate data from a user, utilized within the system for estimating maximal oxygen uptake during physical activity (shown in FIGS. 1-2) according to embodiments of the present disclosure. This flow chart corresponds to block 330 of FIG. 3.

FIG. 7 is a flow chart illustrating a sub-method, for acquiring and storing biomechanical sensor data from a user, utilized within the system for estimating maximal oxygen uptake during physical activity (shown in FIGS. 1-2) according to embodiments of the present disclosure. This flow chart corresponds to block 335 of FIG. 3. FIG. 7 is also a flow chart illustrating a sub-method, for acquiring and storing geophysical data from a user, utilized within the system for estimating maximal oxygen uptake during physical activity (shown in FIGS. 1-2) according to embodiments of the present disclosure. This flow chart corresponds to block 340 of FIG. 3.

FIG. 8 is a flow chart illustrating a sub-method, for determining the maximal heart rate of a user, utilized within the system for estimating maximal oxygen uptake during physical activity (shown in FIGS. 1-2) according to embodiments of the present disclosure. This flow chart corresponds to block 345 of FIG. 3.

FIG. 9 is a flow chart illustrating a sub-method, for computing and storing the oxygen consumption of a user at a succession of time points, utilized within the system for estimating maximal oxygen uptake during physical activity (shown in FIGS. 1-2) according to embodiments of the present disclosure. This flow chart corresponds to block 350 of FIG. 3. FIG. 9 is also a flow chart illustrating a sub-method, for constructing a time series of numerical tuples containing the heart rate and computed oxygen consumption data of a user at a succession of time points, utilized within the system for estimating maximal oxygen uptake during physical activity (shown in FIGS. 1-2) according to embodiments of the present disclosure. This flow chart also corresponds to block 350 of FIG. 3.

FIG. 10 is a flow chart illustrating a sub-method, for extracting from the data collected in the sub-method described in FIG. 9 an estimate of maximal oxygen uptake; the sub-method of this figure is in turn utilized within the system for estimating maximal oxygen uptake during physical activity (shown in FIGS. 1-2) according to embodiments of the present disclosure. This flow chart corresponds to block 355 of FIG. 3.

### DETAILED DESCRIPTION

The present disclosure is directed to systems and methods for determining the aerobic capacity (also known in the art as “\dot{V}O₂max,” “maximal oxygen consumption,” “maximal oxygen uptake,” or “peak oxygen uptake,” among other terms; according to some definitions in the art there are subtle differences among some of the aforementioned terms, but the systems and methods disclosed here apply to all commonly used definitions of these terms) of an individual or group of individuals, typically one or more human beings.

Many potential applications of quantitative techniques for assessing cardiovascular fitness using the “natural experiment” paradigm are not adequately addressed by state-of-the-art methods. Notable examples include:

A. Monitoring the aerobic capacity of casual exercisers and competitive athletes in response to aerobic training over time, in natural (non-laboratory) settings, conveniently, inexpensively, and in a non-effort-dependent manner, in order to determine the effectiveness of their fitness and training regimens.

B. Quantitatively assessing the aerobic capacity of competitive athletes in the competitive setting (rather than in a physiological laboratory), so as to optimize performance strategies for competition.

C. Monitoring or serially measuring the aerobic capacity of individuals known to be at risk for complications of cardiovascular disease (especially individuals with conditions for which aerobic capacity is known to predict morbidity and mortality), in the outpatient setting and without interrupting their activities of daily living, without requiring such individuals to return repeatedly to a specially equipped testing facility.

To address shortcomings in the prior art, the present disclosure introduces a new system and set of methods for assessing the aerobic capacity of an individual or plurality of individuals, using data collected from sensors that monitor heart rate, spatial location, and, optionally, other parameters. The system and methods described here have several principal advantages:

1. Aerobic capacity and related parameters can be quantitatively assessed and monitored serially, over time, at negligible marginal cost per incremental assessment.

2. Aerobic capacity and related parameters can be assessed in “natural” environments, during routine activities, rather than under “laboratory” conditions, which require the use of specialized testing facilities and personnel, and which may imperfectly approximate the conditions of interest (such as field or terrain conditions for an athlete, or activities of daily living for a cardiac patient).

3. Because the method disclosed here includes a model of cardiovascular dynamics, assessment of aerobic capacity and other parameters can be performed dynamically, without the use of specialized, timed protocols designed to bring the cardiovascular system of an individual through a series of steady-state plateaus at increasing intensities.

4. Aerobic capacity can be assessed and monitored over time in larger populations of individuals than would be possible with traditional techniques requiring skilled technicians and specialized equipment.

5. Individuals can easily monitor the effect of training regimens on their aerobic capacity and compare their overall aerobic capacity and rate of improvement to similar age- and gender-matched controls.

6. The low cost of this system and method will bring aerobic capacity estimation to many more individuals than have historically had access to standard laboratory testing.

Exploring each of these advantages in turn, consider the case in which an individual at elevated risk of heart disease, perhaps as a result of moderate obesity, high cholesterol, or a history of having smoked cigarettes in the past, wishes to undertake a program designed to reduce his or her risk of premature death. The user would first provide basic anthropomorphic and biographical data, such as his or her height, weight, and age, as well as pertinent information relating to habits and lifestyle. The user would then conduct a sub-maximal exercise routine at a safe and comfortable intensity level, during which time the system would record geophysical data such as position, velocity, and acceleration and biophysical data such as heart rate. From this data, an initial estimate of his or her aerobic capacity would be obtained, and the system would provide the user with a probability of his or her own chance of premature death using population data freely available in the published scientific governmental literature. The user would then be presented with a series of lifestyle and behavior modifications and their projected impact on aerobic capacity and probability of premature death, derived from both empirical population data as well as model-based simulations. The user would repeat the measurements at frequent intervals to receive feedback that would encourage further compliance and commitment to any chosen behavioral modification program.

Next, consider the case of a casual weekend exerciser, who already has an established exercise routine and may be contemplating any of a number of specific fitness goals, such as losing a certain amount of weight or completing a race in a particular time. The user would first provide basic anthropomorphic and biographical data, such as his or her height, weight, age, and current exercise habits. The system would then prompt the user to define a goal in precise and quantitative terms, such as “to lose 10 pounds in the next 4 months” or “to run 26.2 miles in less than 3 hours and 30 minutes on a date six months from now.” The user would then conduct a baseline exercise routine over a range of safe and comfortable intensity levels, during which time the system would record geophysical data such as position, velocity, and acceleration, and biophysical data such as cadence, respiratory rate, heart rate, and sweat rate. From this data, the aerobic capacity of the user would be estimated, along with an assessment of the present and future ability of the user to achieve the defined goal. The user would then be presented with the probability of meeting that objective, as estimated from model-based estimates of his or her current planned dietary and exercise regimen and empiric comparison to age- and gender-matched controls, as well as recommendations for specific interventions that would increase the likelihood of achieving his or her goal. These interventions may include specific dietary regimens (daily caloric intake, or a specific carbohydrate-loading schedule) or training programs scientifically and empirically demonstrated to increase the probability of success. Because the present system is convenient to use not only on a one-time but also on a repeated basis, the user would be able to receive regular updates over the course of his or her training program, and could watch his or her probability of success increase or decrease over time. In addition, the user could compare his or her progress to that of demographically-matched controls.

Finally, consider the specific example of a professional athlete or team of athletes engaged in competition at the extremes of human performance. Even if such individuals already have access to laboratory-based measurements of maximum aerobic capacity, the present system could still be employed to competitive advantage by allowing for more frequent measurements of exercise capacity in a setting identical or closely approximating actual competitive conditions. Such measurements could potentially be used to monitor the effectiveness of training programs, to predict when an individual athlete needs rest from training, or to design nutritional and strength-building strategies optimized to the unique biochemical and physiologic needs of each individual athlete. Further applications to real-time competitive performance, where the rules and regulations governing the specific sport or event allow, are possible. In this example, an athlete or team of athletes would each be connected to the system via a set of communication-capable sensors that would track and transmit geophysical, environmental, and biophysical measurements in real-time. From such data feeds and stored anthropometric and biophysical measurements of the same individuals, the system would monitor the physiologic status of each athlete over time. Depending on the sport, the system could be used on an individual basis to provide feedback on pacing or optimal nutrition and fluid replacement strategies, or on a team basis to optimize personnel management based on the predicted effectiveness of each athlete at a given point in time.

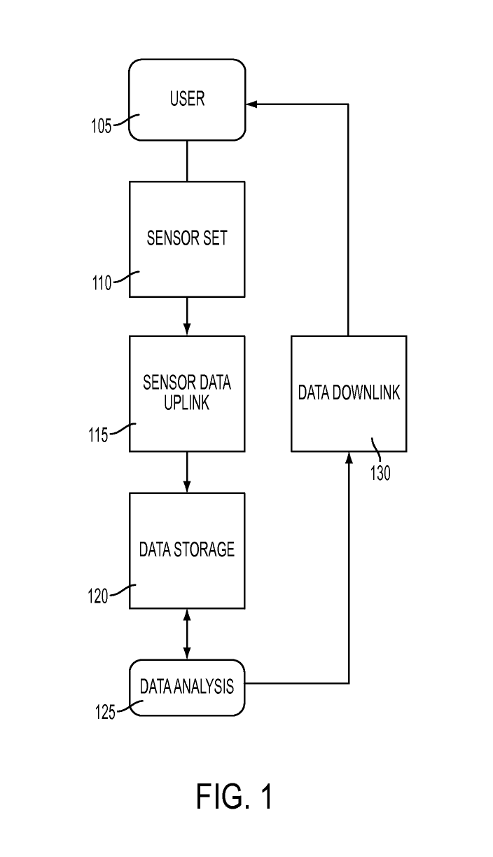

Turning to the drawings, FIG. 1 provides an overview of the system architecture. The system includes a number of sensors 110 that collect information about each user 105 of the system. As will be described in detail in the text that follows, the sensors most importantly include heart rate monitors, global positioning system (GPS) transponders, and accelerometers. The system is in principle compatible with any type of wearable sensor that tracks these parameters (use of other types of sensor is envisioned as well).

The sensors 110 in turn transmit the information they collect from each user 105 to a data storage subsystem 120 through a sensor data uplink 115.

A data analysis subsystem 125 has continuous access to the data accumulated in the data storage subsystem 120, and continuously performs computations as diagrammed in subsequent figures and as described in further detail herein, using data obtained from the sensors mentioned in the previous paragraph to estimate oxygen uptake by the individual user wearing the sensors. The results of these computations may be stored in the data analysis subsystem 125, and may also be transmitted to the original user 105 through a data downlink 130.

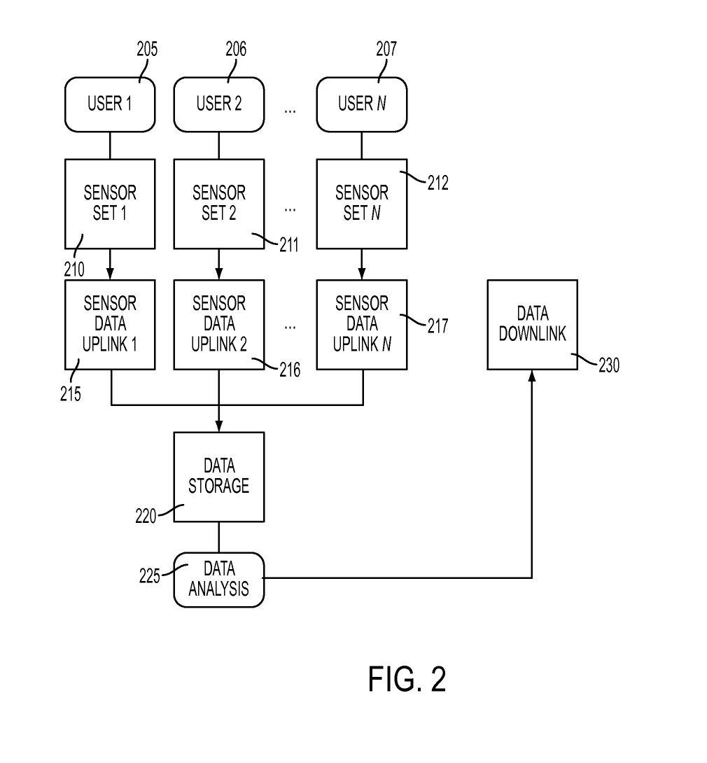

FIG. 2 provides an overview of the system architecture as it may be implemented for multiple users (205, 206, 207, . . . ). As in FIG. 1, which describes the case of a single user 105, multiple users (205, 206, 207, . . . ) are each monitored by corresponding sets of sensors (210, 211, 212, . . . ). Each set of sensors (210, 211, 212, . . . ) uses a corresponding data uplink (215, 216, 217, . . . ) to transmit information collected from its corresponding user (205, 206, 207, . . . ). This transmission may take place in real time or after a time delay following data collection by the sensors. Data from all data uplinks (215, 216, 217, . . . ) are transmitted to and stored in a central data storage subsystem 220. As in the single-user case described in FIG. 1, a data analysis subsystem 225 has continuous access to the data accumulated in the data storage subsystem 220, and continuously performs computations as diagrammed in subsequent figures and as described in further detail herein. The results of these computations may be stored in the data analysis subsystem 225, and may also be transmitted to the users (205, 206, 207, . . . ) through a data downlink 230. In the multi-user case described in FIG. 2, data and computations derived from each user (205, 206, 207, . . . ) may be available to other users (205, 206, 207, . . . ). Data transmission to the users (205, 206, 207, . . . ) through the data downlink 230 may take place in real time or after a time delay, and communication with users (205, 206, 207, . . . ) may take place synchronously or asynchronously; that is, different users (205, 206, 207, . . . ) may receive data simultaneously or at different times, and different users (205, 206, 207, . . . ) may receive identical or different data, depending on the situation.

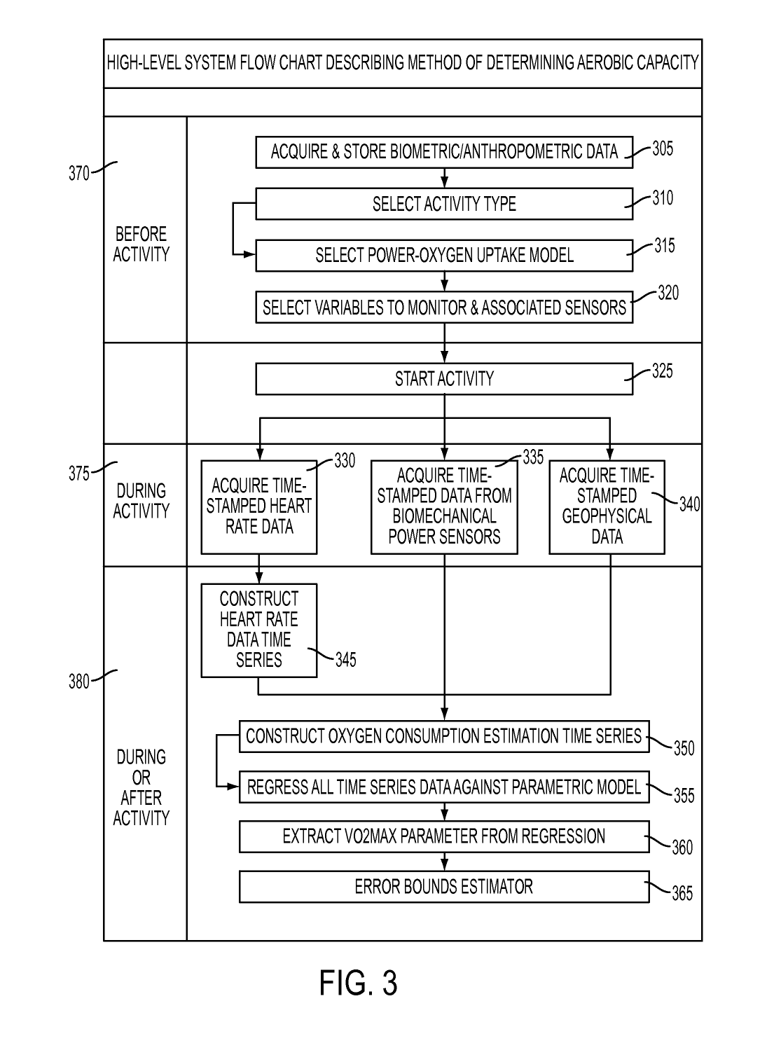

FIG. 3 provides an overview description of the process by which aerobic capacity and other parameters of interest are computed from sensor data acquired from users. Individual components of this process are diagrammed in subsequent figures and as described in further detail herein.

The first phase of the process of estimating aerobic capacity and other parameters of interest begins before any activity commences; parameters of interest include caloric expenditure (partitioned specifically into the fractions of total energy expended through fat metabolism and carbohydrate metabolism), as well as water and electrolyte losses during exercise, among other quantities. This phase is designated “Before Activity” 370, and begins with the acquisition and storage of biometric, anthropometric, and environmental data 305 from each user. Baseline biometric data include weight, age, gender, and height, among other parameters. Anthropometric data include percentage body fat and lean body mass. Environmental data include temperature, humidity, precipitation, barometric pressure, and terrain type (paved road versus trail), among other parameters. The type of activity in which each user is engaged is then identified 310, either through explicit specification by the user or potentially through another scheme. Types of physical activity supported by this system include essentially any activity in which energy consumption is primarily aerobic, and specifically include, but are not limited to, running, walking, hiking, cycling, cross-country skiing, and swimming. The selection of physical activity type 310 leads, in turn, to the selection of a corresponding mathematical model, referred to herein as a “Power-Oxygen Uptake Model” 315, that relates variables monitored by a set of sensors 320 to mechanical and metabolic power output by a user of the system.

The second phase of the process of estimating aerobic capacity and other parameters of interest, designated “During Activity” 375, begins after a user has started to engage in physical activity (a point in the method labeled “Start Activity” 325).

In this second, “During Activity” phase 375, three processes proceed in parallel: “Acquire Time-Stamped Heart Rate Data” 330, “Acquire Time-Stamped Data from Biomechanical Power Sensors” 335, and “Acquire Time-Stamped Geophysical Data” 340. Each of these processes is discussed in detail in reference to subsequent figures. Collectively, these processes provide for the acquisition of a sensor data time series corresponding to each of the variables in the “Power-Oxygen Uptake Model” 315 specified earlier in the process.

The third phase of the process of estimating aerobic capacity and other parameters of interest, designated “During or After Activity” 380, may take place either in parallel with (“During”) or subsequent to (“After”) the second (“During Activity” 375) phase begins after a user has started to engage in physical activity (a point in the method labeled “Start Activity” 325).

In the first step of this phase, “Construct Heart Rate Data Time Series” 345, heart rate data, which, in practice, are collected and reported by many sensors at a sampling rate atypical of biomechanical and geophysical sensors (such as approximately 8 Hz, or twice the maximum physiologic heart rate of approximately 220 beats per minute) may be resampled to construct a data time series equal in dimension to those generated by the other sensors in the system. This resampling may be accomplished through any of various digital resampling techniques well known in the art. In some embodiments, this step may be omitted, while in other embodiments, a similar step may be added for “Acquire Time-Stamped Data from Biomechanical Power Sensors” 335 and “Acquire Time-Stamped Geophysical Data” 340.

The next step in this phase is “Construct Oxygen Consumption Estimation Time Series” 350, in which the selected “Power-Oxygen Uptake Model” 315 is applied to each of the sensed variables, and the resulting estimates of oxygen consumption are paired temporally (by matching time stamps) with corresponding values from the heart rate data generated by “Construct Heart Rate Data Time Series” 345.

Next, the resulting oxygen consumption data series is regressed using one or more methods of statistical regression well known to those skilled in the art, in the step labeled “Regress All Time Series Data Against Parametric Model” 355. The parametric model to which this step refers is a mathematical model relating heart rate and other physiologic parameters to oxygen uptake and aerobic capacity.

In one embodiment, this mathematical model may have the following form:

dR(t)/dt=k(RₘₐₓP_{demand}(t)/\dot{V}O₂ₘₐₓ−R(t)), [Eqn. 1]

where the term R(t) denotes heart rate at time t, the variable k denotes a relaxation constant (which, like \dot{V}O₂ₘₐₓ, may differ from person to person) that describes the rate at which heart rate responds to changes in the overall demand for oxygen by body tissues, Rₘₐₓ denotes the maximum heart rate of the user whose data is being analyzed, P_{demand}(t) denotes the power demanded at a given time, in terms of tissue oxygen uptake, in order to support the sensed biomechanical activity under the sensed geophysical conditions at the corresponding time. Other such parametric models may be used as well.

According to the model described by equation [Eqn. 1] the rate at which heart rate accelerates or decelerates, dR(t)/dt, is proportional to the difference between the aerobic power supplied at the present heart rate, and the power demanded by the body at the current level of exertion. When current aerobic demand exceeds supply, heart rate increases, and dR(t)/dt is positive; when demand falls short of supply, heart rate decreases, and dR(t)/dt is negative; when demand equals supply, steady state is reached, and dR(t)/dt vanishes to zero. The proportionality constant between supply-demand mismatch and change in heart rate is denoted by k; it is equal to 1/τ_{HR} the reciprocal of τ_{HR}, the heart rate relaxation time (which is also equivalent to the half-life for changes in heart rate divided by the natural logarithm of 2).

The equation [Eqn. 1] describes a relationship among parameters, some of which (such as heart rate) are directly measurable, and others of which are inferred from measurable quantities such as P_{demand}(t), which is inferred from activity-dependent parameters, such as speed, incline, and body mass in a runner or walker, that may be used to determine the oxygen demand of the body during exercise according to known relations (Margaria, R., et al., Energy Cost of Running, Journal of Applied Physiology, Vol. 18, pp. 367-370 (1963)). Rₘₐₓ, the maximum heart rate of a particular user, may either be estimated according to any of several estimation formulas described in the literature (Fox, S. M. and Naughton, J. P., Physical Activity and the Prevention of Coronary Heart Disease, Preventive Medicine, Vol. 1, pp. 90-120 (1972); Tanaka, H., et al., Age-Predicted Maximal Heart Rate Revisited, Journal of the American College of Cardiology, Vol. 37(1), pp. 153-156 (2001)), or extracted from direct observation of the particular user over time, as described herein.

The final two parameters referenced in Equation [Eqn. 1], \dot{V}O₂ₘₐₓ and k, are determined by statistical regression in the system described herein. In particular, each time point at which data are collected by the system generates a set of numeric values for R(t), dR(t)/dt, and P_{demand} (t). Thus, at each time point, Equation [Eqn. 1] reduces to a numeric relationship between k and \dot{V}O₂ₘₐₓ. A variety of statistical techniques well known in the art may then be used to estimate the values of these two parameters, together with error bounds around the corresponding estimates.

The next steps in this phase of the process of estimating aerobic capacity are, respectively, to “Extract \dot{V}O₂ₘₐₓ Parameter from Regression” 360 and “Error Bounds Estimator” 365, which will be evident to those skilled in the art as following naturally from the regression step 355. Here “\dot{V}O₂ₘₐₓ” corresponds to the aerobic capacity, and the quantity designated \dot{V}O₂ₘₐₓ in the mathematical model.

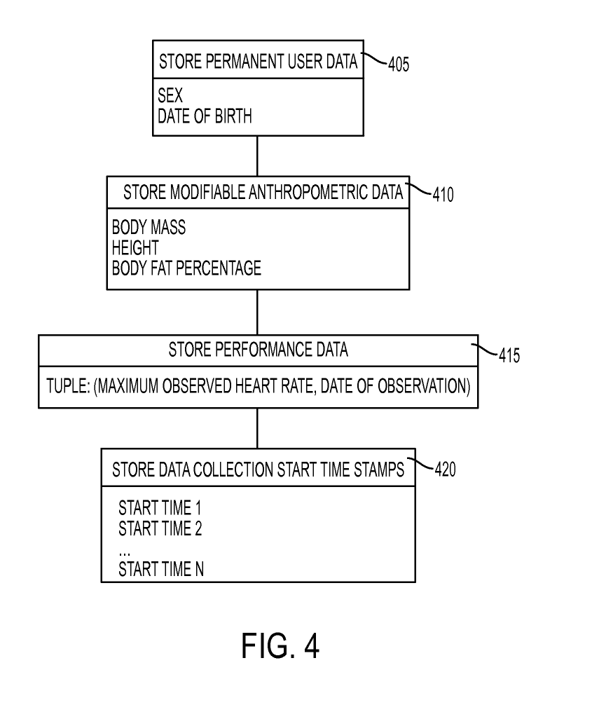

FIG. 4 describes in detail the process labeled “Acquire & Store Biometric/Anthropometric Data” 305 in FIG. 3. In this process, several types of data are acquired from the user.

One type of data, “Permanent User Data” 405, includes variables such as sex and date of birth. Other variables may also be included in this class. The values of these variables are transmitted via the data uplink 215 and are stored in a database of “Permanent User Data” within the main data storage subsystem 220.

A second type of data, “Modifiable Anthropometric Data” 410, includes variables such as body mass, height, and body fat percentage. Other variables may also be included in this class. The values of these variables are transmitted via the data uplink 220 and are stored in a database of “Modifiable Anthropometric Data” within the main data storage subsystem 220.

A third type of data, “Performance Data” 415, includes data describing important features of past data observed for a given user or plurality of users. This type of data includes records of the maximum observed heart rate for a given user, his or her maximum running or cycling speed over particular benchmark distances such as 100 or 1000 meters, his or her maximum achieved \dot{V}O₂ and the time period over which it was sustained, together with the date and time at which such observations are made. These records are updated as appropriate through the computations of the data analysis subsystem 225, and are stored in the data storage subsystem 220.

A fourth type of data, “Data Collection Start Time Stamps” 420, includes the start times for all periods during which data were collected from a given user. These time stamps are established each time the “Start Activity” 325 step of the method is reached; they are transmitted via the data uplink 215 and are stored in a database of “Data Collection Time Stamps” within the main data storage subsystem 220.

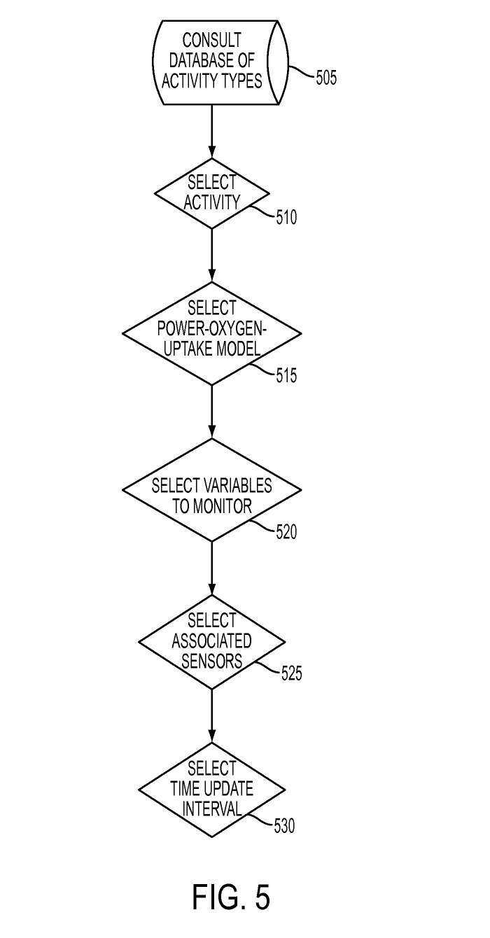

FIG. 5 describes in detail the processes labeled “Select Activity Type” 310 in FIG. 3. In this process, the system matches the form of physical activity in which each user is engaged with a set of variables to be monitored during that activity, and also with a mathematical model relating the values of those variables to the oxygen consumption rate of the user while engaged in that activity.

The selection of the mathematical model is indicated by the step “Select Power-Oxygen Uptake Model” 515. The selection of the variables to be monitored by sensors is indicated by the step “Select Variables to Monitor and Associated Sensors” 320.

The “Select Activity Type” 310 process begins with the step “Consult Database of Activity Types” 505. In this step, a “Select Activity” 510 decision is made, in which a type of physical activity (such as, for example, walking, hiking, running, cycling, rowing, or any of a wide variety of possible forms of physical activity) is selected corresponding to the type of physical activity in which the user is about to engage.

Following this initial selection, a “Select Power-Oxygen-Uptake Model” 515 decision is made, either automatically by the system (using a default, demographics-based, or other basis for selection) or by the user, as to the appropriate mathematical model relating monitored parameters to oxygen uptake during physical activity.

Implicit in the “Select Power-Oxygen-Uptake Model” 515 decision is another decision, “Select Variables to Monitor” 520, which designates the variables for which data will be collected from sensors monitoring aspects of user physical activity. The “Select Associated Sensors” 525 decision can then be made either automatically (using programmed defaults, detection of user devices connected to the system, or through other means) or through explicit input from one or more users; in this step, sensors whose data streams will be processed by the system as user activity is monitored are registered for use by the system. Data from many types of sensors may be used; basic examples of important sensors include heart rate monitors, accelerometers, gyroscopes, and Global Positioning System (GPS) transponders. In most contemplated applications, heart rate and position data will be required.

The final decision in this step is “Select Time Update Interval” 530, in which a global time step, Δt, is designated for data acquisition. Synchronous, asynchronous, and hybrid schemes for data collection may be used.

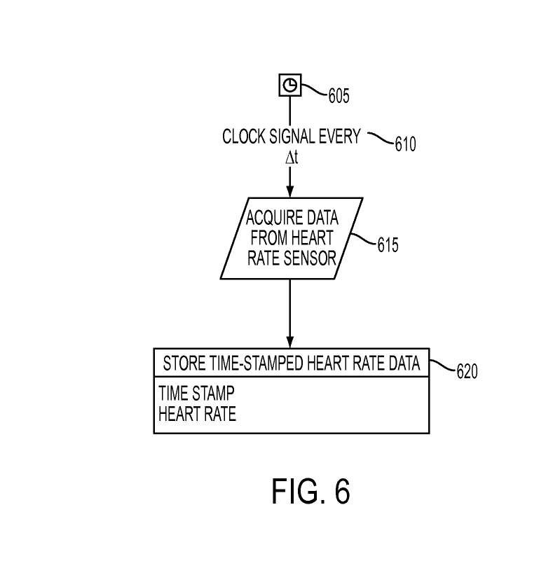

FIG. 6 describes in detail the process labeled “Acquire Time-Stamped Heart Rate Data” 330 in FIG. 3. In this process, the system acquires heart rate data from a heart rate sensor worn by a user. A heart rate sensor worn by a user generates heart rate data, typically at time intervals Δt measured by a Clock 605 that generates a periodic “Clock Signal Every Δt” 610 (though other datastream paradigms are conceivable, including variable sampling intervals as might be the case in which data consist of heart beat time stamps). As indicated by “Acquire Data from Heart Rate Sensor” 615, the heart rate data generated by the heart rate sensor are acquired, then stored in a database, as indicated by “Store Time-Stamped Heart Rate Data” 620; the stored data entries consist, minimally, of heart rate values and their corresponding times of acquisition (which are designated here, consistent with common practice, as “time stamps”).

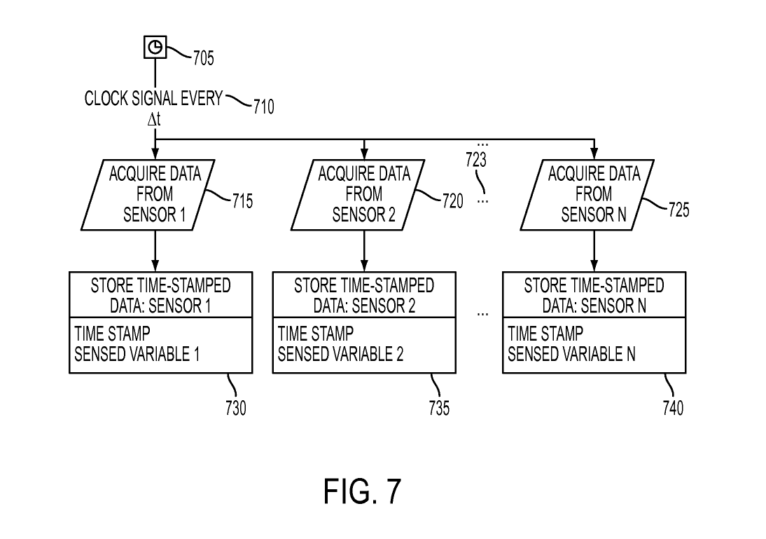

FIG. 7 describes in detail the processes labeled “Acquire Time-Stamped Geophysical Data” 340 and “Acquire Time-Stamped Data from Biomechanical Power Sensors” 335 in FIG. 3. These processes function in parallel with and are similar to “Acquire Time-Stamped Heart Rate Data” 330 of FIG. 3, as it is described in detail in FIG. 6. They employ the same Clock 705=605 that generates a periodic “Clock Signal Every Δt” 710=610 that is used to synchronize sensor data acquisition and to label time stamps (though other datastream paradigms are possible, including variable sampling intervals). The processes “Acquire Time-Stamped Geophysical Data” 340 and “Acquire Time-Stamped Data from Biomechanical Power Sensors” 335 operate simultaneously, in parallel, and according to the following data acquisition scheme; they differ essentially only in the sensors from which they acquire data. As indicated by the data acquisition modules, “Acquire Data from Sensor 1” 715, “Acquire Data from Sensor 2” 720, and “Acquire Data from Sensor N” 725, both geophysical and biomechanical power data are acquired from sets of multiple sensors, simultaneously and in parallel. The number of sensors can range from 1 to N, an arbitrarily large number (the ellipsis 723 stands for parallel sensor and data storage modules identical to those numbered 1, 2, and N). In the case of geophysical data sensors, sensed variables may include GPS coordinates (latitude, longitude, altitude), three dimensions of velocity, and three dimensions of acceleration; variables conveying information related to temperature, barometric pressure, wind speed and direction, terrain type; as well as other variables. In the case of biomechanical data, sensed variables may include cadence (as in the stride rate of a runner, or the pedaling rate of a cyclist), running stride length, and many other possible measurable parameters related to body movement during exercise. As indicated by the data storage modules, “Store Time-Stamped Data: Sensor 1” 730, “Store Time-Stamped Data: Sensor 2” 735, and “Store Time-Stamped Data: Sensor N” 740, both geophysical and biomechanical power data are stored, once acquired from their associated sensors, in corresponding databases. The stored data entries consist, minimally, of the values of the sensed variables and their corresponding times of acquisition (time stamps).

In practice, sensors acquire data together with significant amounts of noise. The effects of sensor noise on computations and overall system function can be reduced by using inherent correlations among multiple independent data streams to distinguish signal from noise with statistical confidence. For example, because running speed and heart rate are known to be tightly correlated for physiologic reasons, an abrupt change in heart rate registered by a heart rate monitor that is not correlated with a change in speed as measured by GPS may be classified as noise; this determination could be established with greater confidence if an additional sensor, such as an accelerometer or other instrument measuring running cadence, registers no change in cadence during the same interval. Formal signal processing techniques, including but not limited to Kalman filtering, may be employed to implement such denoising schemes, and such post-processing schemes may optionally be implemented after sensor data are stored.

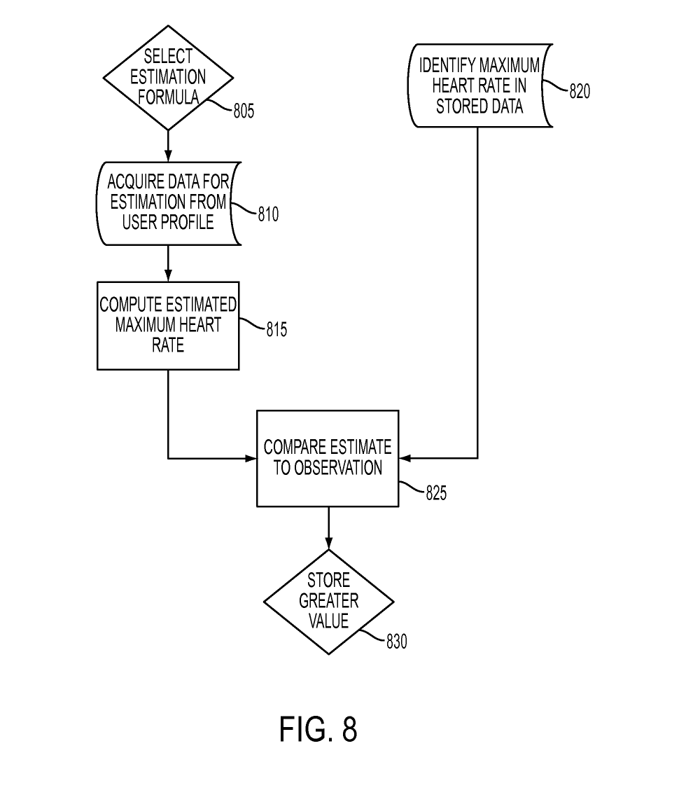

FIG. 8 describes a process for estimating the maximal heart rate of a user. The maximum heart rate of a user is used as a parameter in estimating other quantities, including the maximal oxygen uptake, as reflected by the appearance of the parameter Rₘₐₓ in Eqn. 1. The estimation method described here begins with “Select Estimation Formula” 805, a step in which a particular regression formula is selected on the basis of which maximal heart rate may be estimated from demographic parameters such as age and gender, possibly supplemented by additional data. Many such regression formulas have been described in the scientific literature and are known in the art, and their predictions may guide initial estimations even though the accuracy of such formulas is known to be limited. Note, however, that the current method and system are designed to accumulate data from large populations of users, which will facilitate greater statistical accuracy in estimation of maximum heart rate values. In the step “Acquire Data for Estimation from User Profile” 810, the demographic data required by the selected maximal heart rate estimation formula are retrieved from the “Permanent User Data” database 405. In the step “Compute Estimated Maximum Heart Rate” 815, the data acquired from the Permanent User Data database are entered into the chosen formula for estimating maximum heart rate to obtain an estimate of the maximum heart rate of a particular user. Simultaneously, in the step “Identify Maximum Heart Rate in Stored Data” 820, the system searches the “Performance Data” 415 database for the maximum heart rate achieved by the user in question. Then, in the step “Compare Estimate to Observation” 825, the estimate of maximum heart rate obtained in the “Compute Estimated Maximum Heart Rate” step 815 is compared to the highest heart rate that the system has observed for the user in question. As indicated by “Store Greater Value” 830, the greater of these two values is stored by the system as its estimate of the maximum heart rate for the user in question.

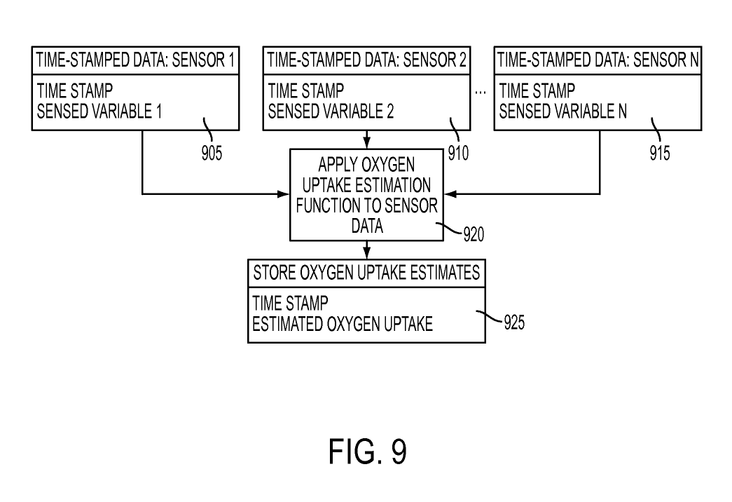

FIG. 9 describes in detail the process labeled “Construct Oxygen Consumption Estimation Time Series” 350 in FIG. 3. In this step, the selected “Power-Oxygen Uptake Model” 315 is applied to each of the sensed variables, and the resulting estimates of oxygen consumption are paired temporally (by matching time stamps) with corresponding values from the heart rate data generated by “Construct Heart Rate Data Time Series” 345. More specifically, the sensed variables in blocks “Time-Stamped Data: Sensor 1” 905, “Time-Stamped Data: Sensor 2” 910, and “Time-Stamped Data: Sensor N” 915 correspond to the modules “Store Time-Stamped Data: Sensor 1” 730, “Store Time-Stamped Data: Sensor 2” 735, and “Store Time-Stamped Data: Sensor N” 740 of FIG. 7. “Sensed Variable 1” and the other sensed variables, through “Sensed Variable N,” correspond to the biometric and geophysical parameters monitored for each particular user (N can differ from user to user), as described earlier in reference to FIG. 7. The multiple data series from the set of sensors used by a given user are entered into the designated “Power-Oxygen Uptake Model,” as indicated by the step “Apply Oxygen Uptake Estimation Function to Sensor Data” 920, and the resulting computed results, paired with associated time stamps, are stored in an “Oxygen Uptake Estimates” database, as indicated by the module “Store Oxygen Uptake Estimates” 925.

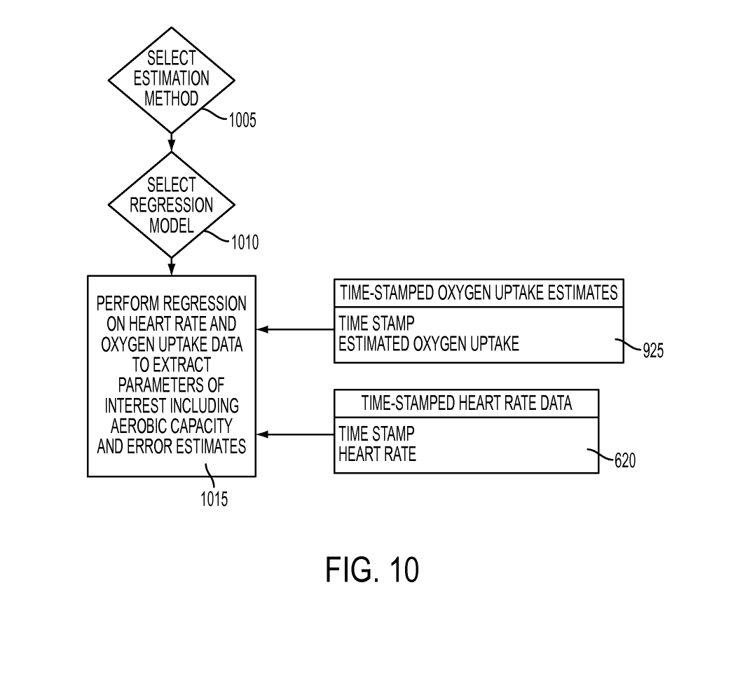

FIG. 10 describes in detail the processes labeled “Regress All Time Series Data Against Parametric Model” 355, “Extract \dot{V}O₂ₘₐₓ Parameter from Regression” 360, and “Error Bounds Estimator” 365 in FIG. 3. As indicated by “Select Estimation Method” 1005, this process begins by selecting a set of statistical techniques, from among the many described in the art, to use in estimating parameters of interest, such as aerobic capacity, from the data series collected from a particular user. Estimation methods include Bayesian, parametric, and other statistical techniques. Bayesian methods may in general be preferred, and have been used in the initial reduction to practice of the methods described here, as they facilitate implementation of explicit prior constraints based on the known population distribution of physiologic parameters such as \dot{V}O₂ₘₐₓ, and the known physiologic range for heart rate relaxation, k.

As further indicated by “Select Regression Model” 1010, the next step of this process is to define a relationship among the variables whose values are reflected in the collected data, and parameters of interest, including aerobic capacity. One such relationship is defined in Eqn. 1; other such relationships are possible. Finally, the system performs the step designated “Perform Regression on Heart Rate and Oxygen Uptake Data to Extract Parameters of Interest Including Aerobic Capacity and Error Estimates” 1015, in which the chosen statistical techniques and regression model are used to extract parameters of interest, including the aerobic capacity of a particular user, together with estimates as to the estimation error. The mathematical details of these methods will be self-evident to those skilled in the art.

This critical step may be clarified through example. Suppose that the system is monitoring a 30-year-old runner of mass 60 kilograms, running at 9 kilometers per hour on level ground but gradually increasing her speed. Suppose further that her present heart rate is 150 beats per minute the system finds her heart rate to be accelerating at a rate of dR(t)/dt equal to 1 beat per minute per minute. The calculated maximum heart rate of this runner by the method of Fox and Haskell is 190 beats per minute; if the system has recorded a maximum observed heart rate different from 190 it may preferentially use the value it has stored (as described in FIG. 8) as Rₘₐₓ. The aerobic power demanded at this level of exertion is approximated (Glass, S., et al., ACSM's Metabolic Calculations Handbook, Lippincott Williams & Wilkins (2007)) by

\dot{V}O₂=0.2×s+0.9×s×g+3.5

where s denotes running speed in meters per minute and g denotes the fractional grade of any vertical incline in the running path (which is zero in this example on level ground). In the present example, therefore, P_{demand}(t) is calculated to be 33.5 mL O₂ min⁻¹ Using Eqn. 1, substituting the values just derived provides the following numeric relationship at the present time point, t:

1=k(190×33.5/\dot{V}O₂ₘₐₓ−150).

As this example demonstrates, a mathematical model such as the one presented in Equation [Eqn. 1] gives rise to a numerical relationship between the physiologic parameters k (heart rate relaxation time constant) and \dot{V}O₂ₘₐₓ (maximum oxygen uptake) at every time point. By collecting many such relationships at multiple time points throughout an observational interval, the system can use standard statistical regression techniques for estimating values of \dot{V}O₂ₘₐₓ and k, together with error bounds on the associated estimates.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of nonvolatile memory, including by way of example semiconductor memory devices, (e.g., EPROM, EEPROM, and flash memory devices); magnetic disks, (e.g., internal hard disks or removable disks); magneto optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back end component (e.g., a data server), a middleware component (e.g., an application server), or a front end component (e.g., a client computer having a graphical user interface or a web browser through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods, and systems for carrying out the several purposes of the disclosed subject matter. It is important, therefore, that the claims be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter, which is limited only by the claims which follow.

1. A computerized method for dynamically measuring maximal oxygen uptake of a user in real-time during aerobic activity, the method comprising:
(a) electronically measuring instantaneous heart rate data, instantaneous biomechanical data, and instantaneous geophysical data of the user over a period of time, using one or more sensors;
(b) setting an oxygen uptake model for the user and storing the oxygen uptake model in memory of a computer;
(c) determining, using the computer, a maximum heart rate of the user and storing the maximum heart rate in memory;
(d) determining, using the computer, a plurality of instantaneous oxygen uptake estimates over the period of time based in part on user data including the maximum heart rate, the instantaneous biomechanical data, and the instantaneous geophysical data, wherein the user data is selected and related to the plurality of instantaneous oxygen uptake estimates using the oxygen uptake model;
(e) evaluating, using the computer, a relationship between a real-time heart rate relaxation constant and a real-time maximal oxygen uptake of the user based at least in part on the plurality of the instantaneous oxygen uptake estimates, the maximum heart rate, the instantaneous heart rate data, the instantaneous biomechanical data, and the instantaneous geophysical data, wherein the real-time heart rate relaxation constant comprises a numerical parameter that measures a rate at which the heart rate of a user changes in response to oxygen demand; and
(f) determining, using the computer, a maximal oxygen uptake for the user during the aerobic activity, using the relationship between the real-time heart rate relaxation constant and the real-time maximal oxygen uptake.

2. The method of claim 1, wherein the aerobic activity comprises running, walking, hiking, cycling, cross-country skiing, swimming or stair climbing.

3. The method of claim 1, wherein the user comprises a mammal.

4. The method of claim 1, wherein the user comprises a human.

5. The method of claim 1, wherein the user comprises a horse or a dog.

6. The method of claim 1, wherein determining a heart rate relaxation constant (k) and a maximal oxygen uptake \dot{V}O₂ₘₐₓ, comprises determining the following relationship:
$$\frac{d R ( t )}{d t} = k ( {R}_{ma x} \frac{{P}_{demand} ( t )}{\dot{V} {O}_{2 m ax}} - R ( t ) )$$
wherein R(t) comprises heart rate at a given time, Rₘₐₓ comprises the maximum heart rate, and P_{demand}(t) comprises a power demanded at a given time.

7. The method of claim 1, wherein the biomechanical data comprises instantaneous speed of the user.

8. The method of claim 1, wherein the geophysical data comprises instantaneous incline, latitude, longitude, and altitude data.

9. The method of claim 1, wherein the one or more sensors comprise a heart rate monitor, a global positioning sensor, and a gyroscope.

10. The method of claim 1, comprising repeating steps (a)-(f) over a plurality of periods of time.

11. The method of claim 1, comprising performing steps (a)-(f) for a plurality of users.

12. (canceled)

13. A computerized method for dynamically measuring maximal oxygen uptake of a user in real-time during aerobic activity, the method comprising:
(a) electronically measuring instantaneous physiologic data, instantaneous biomechanical data, and instantaneous geophysical data of the user over a period of time, using one or more sensors;
(b) using a computer to create physiologic, biomechanical, and geophysical time-series data based on the instantaneous physiologic data, instantaneous biomechanical data, and instantaneous geophysical data;
(c) establishing, using the computer, an oxygen uptake model for the user;
(d) estimating, using the computer, a plurality of instantaneous oxygen uptake values over the period of time based in part on user data including the instantaneous physiologic data, the instantaneous biomechanical data, and the instantaneous geophysical data obtained by the sensors, wherein the user data is selected and related to the plurality of instantaneous oxygen uptake values using the oxygen uptake model;
(e) predicting, using the computer, a maximum oxygen uptake of the user based on the oxygen uptake model, the oxygen uptake values and the physiologic, biomechanical, and geophysical time-series data.

14. The method of claim 13 wherein a model relating oxygen uptake to physiologic, biomechanical, and geophysical parameters comprises:
$$\frac{d R ( t )}{d t} = k ( {R}_{ma x} \frac{{P}_{demand} ( t )}{\dot{V} {O}_{2 m ax}} - R ( t ) )$$
wherein R(t) comprises heart rate at a given time, k comprises a heart rate relaxation constant, Rₘₐₓ comprises the maximum heart rate, \dot{V}O₂ₘₐₓ, comprises a maximal oxygen uptake value for the user, and P_{demand}(t) comprises the aerobic power demanded at a given time.

15. The method of claim 14, wherein the heart rate relaxation constant comprises a numerical parameter that measures a rate at which the heart rate of a user changes in response to oxygen demand by body tissues.

16. The method of claim 13, wherein the aerobic activity comprises running, walking, hiking, cycling, cross-country skiing, swimming or stair climbing.

17. The method of claim 13, wherein the user comprises a mammal.

18. The method of claim 13, wherein the user comprises a human.

19. The method of claim 13, wherein the user comprises a horse or a dog.

20. The method of claim 13 comprising performing steps (a)-(e) for a plurality of users.

21. The method of claim 13, wherein the biomechanical data comprises instantaneous speed of the user.

22. The method of claim 13, wherein the geophysical data comprises instantaneous incline, latitude, longitude, and altitude data.

23. The method of claim 13, wherein the one or more sensors comprise a heart rate monitor, a global positioning sensor, and a gyroscope.

24. The method of claim 13, comprising repeating steps (a)-(e) over a plurality of periods of time.
